(19) United States
(12) Patent Application Publication
(10) Pub. No.: US 2021/0331156 A1
(43) Pub. Date: Oct. 28, 2021

(54) MULTI-CHAMBER TEST TUBE WITH SELECTIVELY BREACHABLE SEPARATORS

(71) Applicant: William J. Zinnanti, Santa Cruz, CA (US)

(72) Inventor: William J. Zinnanti, Santa Cruz, CA (US)

(21) Appl. No.: 16/916,698

(22) Filed: Jun. 30, 2020

Related U.S. Application Data
(60) Provisional application No. 63/015,998, filed on Apr. 27, 2020.

(51) Int. Cl. B01L 3/00; C12Q 1/686

(52) U.S. Cl. CPC B01L 3/5082; C12Q 1/686; B01L 2300/0848; B01L 2300/0861; B01L 3/5085

(57) ABSTRACT

A multi-chamber test tube and method of using same is provided, wherein the multi-chamber test tube has a selectively breachable internal divider structure. The internal divider structure includes at least one septum that divides an internal volume of the test tube into at least two chambers that are isolated from liquid communication with one another. At least a portion of the at least one septum is configured to be breached under predetermined conditions, to permit liquid communication between the at least two chambers. The multi-chamber test tube has application, for example, during a quantitative or real time-polymerase chain reaction (qPCR or RT-PCR) test, to facilitate accurate detection of gene expression and pathogen detection using RNA analysis by allowing for the initial separation of the reverse transcription reaction from the PCR reaction. Initial reverse transcription of target RNA is completed prior to amplification of the resulting complementary DNA (cDNA) used in the amplification to provide the resulting signal that is quantified.

This application claims priority under 35 U.S.C. § 119(e)(1) of U.S. Ser. No. 63/015,998, filed 27 Apr. 2020 (27.04.2020), the entire contents of which are hereby expressly incorporated by reference.

### TECHNICAL FIELD

The present invention relates to the field of laboratory equipment, specifically, test tubes and similar devices, particularly for use in biological and medical testing and assays, and related procedures.

### BACKGROUND OF THE INVENTION

RT-PCR or qPCR has evolved to provide a useful method to quantitatively measure gene expression or pathogen detection using the combination of three steps including 1) RNA isolation, 2) reverse transcription to form template DNA from RNA known as complementary DNA (cDNA) (FIG. 1) and 3) PCR or amplification of the cDNA. This technology uses specific oligonucleotides or primers with sequence specificity to the target nucleotide code that accurately localizes to one region of the messenger RNA or viral RNA in a highly specific manner. The localization of the primer recruits reverse transcriptase enzyme to create cDNA templates that can later be used for amplification using the same specific primers. After the cDNA templates are made from the isolated RNA, the same specific primers are used to localize to the cDNA sequence. Localization of the primers to the cDNA templates, recruits DNA polymerase to replicate the DNA sequence from both ends by repeatedly cycling the reaction mixture through higher and lower temperatures. Unlike RNA, DNA is double stranded and must be denatured or unzipped to allow primer localization and polymerase recruitment. As the reaction mixture is brought to a lower temperature the polymerase is active and creates copies of the DNA. DNA denatures or unzips at higher temperature (˜95 degree C.) and polymerase is active at lower temperature (˜60 degrees). The reaction mixture is taken through multiple cycles of these two steps resulting in exponential amplification of the DNA. There is a third oligonucleotide in addition to the primers known as a probe. The probe localizes to a specific section in between the forward and reverse primers that encompass the section of DNA being copied. This probe has a fluorescent molecule bonded to one end of the specific sequence and a fluorescent quenching molecule attached to the opposite end. When the two molecules are held in close proximity by the short sequence of specific code, the fluorescence is quenched and not visible. As the polymerase copies the DNA template the probe hydrolyzed releasing the fluorescence from the quenching molecule and the fluorescent signal can be detected (FIG. 2).

Reverse transcriptase and DNA polymerase reactions both require the same nucleotide substrate to make cDNA or copy and amplify DNA. Performing the two reactions in the same reaction mixture causes competition for substrate and interference between the two reactions with resulting loss of sensitivity and efficiency (Al-Shanti et al 2009—Appendix 1 hereto). This principle can affect the accurate analysis of gene expression with analysis of messenger RNA (mRNA) or viral RNA. This interference becomes a dangerous and life-threatening matter when the interference leads to inability to accurately detect pathogens leading to false negative results. This has led to the false assumption that a patient is no longer contagious and release from quarantine results in further infection of others. Additional background information relevant to the instant disclosure may be found in Appendix 1 and Appendix 2 hereto, the complete disclosures of which are hereby expressly incorporated by reference. Appendix 2 was, at the time of this writing, accessible at https://www.thermofisher.com/us/en/home/brands/thermo-scientific/molecular-biology/molecular-biology-learning-center/molecular-biology-resource-library/spotlight-articles/basic-principles-rt-qpcr.html

In recent years technology has developed to speed up testing time without allocating for the interference of these reactions leading to false assumptions about both gene expression and pathogen detection. Separating the reactions, maintains the sensitivity and efficiency of converting the RNA to DNA so that adequate cDNA is provide for the subsequent amplification that provides the resulting quantitated signal.

Currently, disposable test tubes are used in PCR that have a single chamber and therefore two different sets of tubes are required to perform these reactions separately in order to maintain the efficiency and sensitivity for more accurate RNA analysis. This requires additional handling and transfer of reaction mixtures causing increase time for processing. Alternatively, popular 1-step kits are used that combine these reactions together in one mixture into one tube and sacrifice the sensitivity and efficiency of the reactions.

### SUMMARY OF THE INVENTION

Therefore, it is desirable, advantageous and cost saving to have at least a two-chamber test tube where reaction mixtures for the reverse transcriptase and PCR can be held in their respective chambers to allow reverse transcriptase to complete the reaction without interference. This is typically done at 42 degrees C. for 15-20 min. After the completion of this step, the reaction mixture is brought to 95 degree C. to inactivate reverse transcriptase and activate DNA polymerase. It would be advantageous if the separator material, keeping the two mixtures separate, could be destroyed, deformed, lifted or otherize opened at higher temperature (60-95 degree C.). This would allow for the reaction mixtures to combine and allow the product cDNA of the reverse transcriptase reaction access to the PCR mixture at the same time reverse transcriptase is inactivated and DNA polymerase is activated. Therefore, this process or method can maintain the sensitivity and efficiency of the reactions being performed separately.

It is a further advantage of this multi-chamber test tube to be manufactured in strips of 8 individual units or 96 well plates with repeating identical individual tubes for the processing of multiple samples.

It is a further advantage of this multi-channel test tube to be manufactured to fit into available equipment for processing.

It is a further advantage of this multi-channel test tube to have an optically clear top or cap so that fluorescence within the tube can be transmitted to detectors located above the tubes.

The present invention comprises, in part, a multi-chambered test container. An outer shell defines an inner volume. A septum, disposable within the shell, has at least one wall defining at least two chambers within the inner volume. A mechanism is cooperatively engaged with at least one of the outer shell and the septum, which causes a change in relationship between the at least two chambers, such that in a first configuration, the at least two chambers are not in liquid communication to one another, and in a second configuration, the at least two chambers are in liquid communication with one another.

In an embodiment, the mechanism comprises the septum being fabricated from a material that will at least partially fail when the test container is exposed to at least one of a predetermined temperature and a predetermined pressure.

In an embodiment, the mechanism comprises the septum being moved from a first physical orientation relative to the outer shell, wherein the at least two chambers are not in liquid communication, to a second orientation, wherein the at least two chambers are in liquid communication, the movement of the septum occurring when the test container has been exposed to at least one of a predetermined temperature and a predetermined pressure.

In an embodiment, the mechanism comprises a plug, disposed in or adjacent to at least one of the septum and an inner wall of the outer shell, wherein the plug is fabricated from a material that will at least partially fail when the test container is exposed to at least one of a predetermined temperature and a predetermined pressure.

In an embodiment, the mechanism further comprises thermal expansion causing separation of an inner surface of the outer shell and the septum.

In an embodiment, the mechanism comprises a frangible membrane disposed between a lower edge of the septum and a bottom inner surface of the shell.

In an embodiment, the mechanism comprises an expandable chamber which expands upon application of heat to a predetermined temperature and exerts pressure on the septum to dislodge the septum at least partially away from an inner surface of the outer shell.

In an embodiment, the mechanism comprises exposing the test container to vibration to dislodge the septum at least partially away from an inner surface of the outer shell.

In an embodiment, the mechanism comprises the septum being fabricated from a material having a lower coefficient of thermal expansion than the material of the outer shell, such that upon exposure to heat above a predetermined temperature, the septum will become separated from the outer shell.

In an embodiment, the mechanism comprises a pocket, defined between mating portions of the septum and the inner surface of the outer shell, such that upon exposure to heat above a predetermined temperature, gas entrapped within the pocket expands and forces separation of the septum from the outer shell.

The present invention further comprises, in part, a method of performing a test, comprising:

providing a multi-chambered test container, comprising the steps of:
providing an outer shell, defining an inner volume;
providing a septum, disposable within the shell, having at least one wall defining at least two chambers within the inner volume;
providing a mechanism cooperatively engaged with at least one of the outer shell and the septum, which causes a change in relationship between the at least two chambers, such that in a first configuration, the at least two chambers are not in liquid communication to one another, and in a second configuration, the at least two chambers are in liquid communication with one another;

the method further comprising the steps of:

placing at least one first reactant within a first of the defined at least two chambers;

placing at least one second reactant with a second of the defined at least two chambers;

disposing the septum within the shell;

initiating a test procedure using the multi-chambered test container; and

actuating the mechanism.

In an embodiment of the invention, the septum comprises an inner shell, defining an inner shell inner volume, the inner shell being insertingly receivable within at least a portion of the outer shell; and the mechanism is cooperatively engaged with the inner shell.

In an embodiment of the invention, the mechanism comprises:

an aperture disposed in a generally-bottom region of the inner shell; and

a plug, disposed in or adjacent to the aperture, wherein the plug is fabricated from a material that will at least partially fail when the test container is exposed to at least one of a predetermined pressure and a predetermined temperature.

In an embodiment of the invention, the multi-chambered test container further comprises a mechanism for preventing the inner shell from bottoming out in the outer shell.

In an embodiment of the invention, the multi-chambered test container further comprises a mechanism for preventing undesired separation of the inner and outer shells, once the inner shell has been inserted into the outer shell.

In an embodiment of the invention, the mechanism comprises the septum being fabricated from a material that will at least partially fail when the test container is exposed to at least one of a predetermined temperature and a predetermined pressure.

In an embodiment of the invention, the method further comprises the mechanism comprising the septum being moved from a first physical orientation relative to the outer shell, wherein the at least two chambers are not in liquid communication, to a second orientation, wherein the at least two chambers are in liquid communication, the movement of the septum occurring when the test container has been exposed to at least one of a predetermined temperature and a predetermined pressure.

In an embodiment of the invention, the method comprises the mechanism further comprising a plug, disposed in or adjacent to at least one of the septum and an inner wall of the outer shell, wherein the plug is fabricated from a material that will at least partially fail when the test container is exposed to at least one of a predetermined temperature and a predetermined pressure.

In an embodiment of the invention, the method comprising the mechanism further comprising thermal expansion causing separation of an inner surface of the outer shell and the septum.

### BRIEF DESCRIPTION OF THE DRAWINGS

FIG. 1 is a graphic briefly outlining the Reverse Transcription Polymerase Chain Reaction (RT-qPCR).

FIG. 2 is a graphic outlining the process of using a polymerase chain reaction (PCR) with probe primer mechanism to achieve fluorescent signal by amplification of a specific region of DNA of cDNA created from an original RNA target sequence.

FIG. 3 is a schematic illustration of a generalized procedure for the RT-PCR reaction in a laboratory setting.

FIG. 4 is a schematic cross-sectional view of a test tube according to an embodiment of the invention.

FIG. 5 is a schematic cross-sectional view of the test tube according to the embodiment of FIG. 4, orthogonal to the view of FIG. 4.

FIG. 6 is a schematic cross-sectional view of a test tube according to an embodiment of the invention.

FIG. 7 is a schematic cross-sectional view of the test tube according to the embodiment of FIG. 6, orthogonal to the view of FIG. 6.

FIG. 8 is a schematic cross-sectional view of a test tube according to an embodiment of the invention.

FIG. 9 is a schematic cross-sectional view of the test tube according to the embodiment of FIG. 8, orthogonal to the view of FIG. 8.

FIG. 10 is a schematic cross-sectional view of a test tube according to an embodiment of the invention.

FIG. 11 is a schematic cross-sectional view of the test tube according to the embodiment of FIG. 10, orthogonal to the view of FIG. 10.

FIG. 12 is a schematic cross-sectional view of a test tube according to an embodiment of the invention.

FIG. 13 is a schematic cross-sectional view of the test tube according to the embodiment of FIG. 12, orthogonal to the view of FIG. 12.

FIG. 14 is a schematic cross-sectional view of a test tube according to an embodiment of the invention.

FIG. 15 is a schematic cross-sectional view of the test tube according to the embodiment of FIG. 14, orthogonal to the view of FIG. 14.

FIG. 16A is a schematic cross-sectional view of a test tube according to an embodiment of the invention, showing the separate components prior to use.

FIG. 16B is a schematic cross-sectional view of the test tube according to the embodiment of FIG. 16A, subsequent to connection.

FIG. 16C is a schematic cross-sectional view of the test tube according to the embodiment of FIG. 16A, subsequent to connection and actuation of the mechanism for causing a change in the relationship between the two chambers.

FIG. 17 is a schematic cross-sectional view of the test tube according to an embodiment of the invention.

FIG. 18 is a schematic cross-sectional view of the test tube according to an embodiment of the invention.

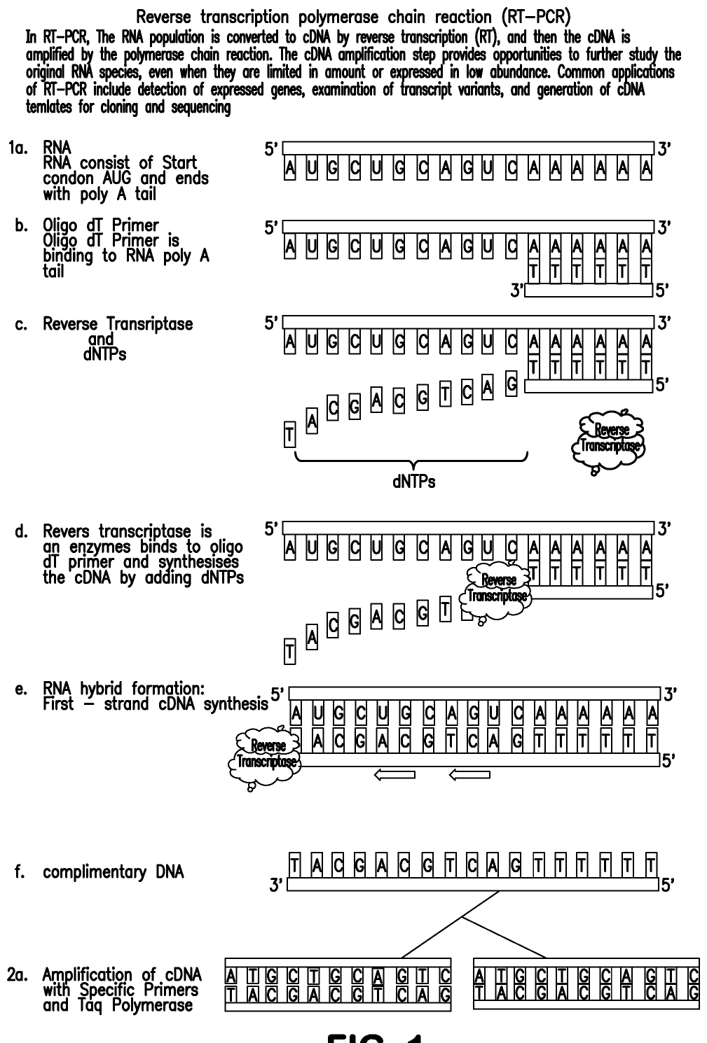

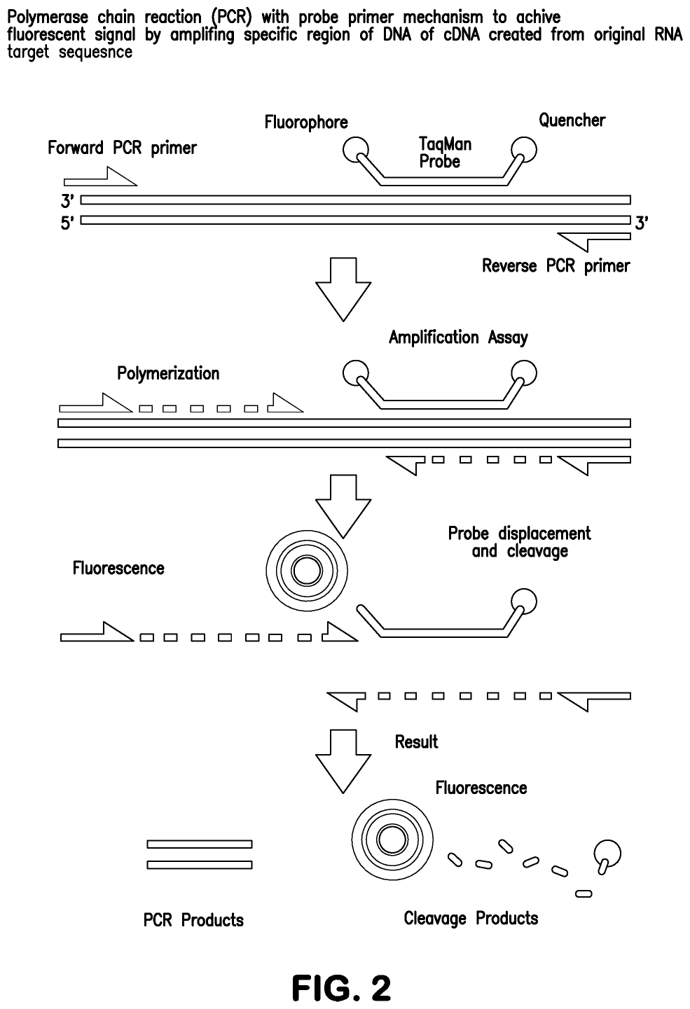

### DETAILED DESCRIPTION OF THE DRAWINGS

While this invention is susceptible of embodiment in many different forms, there are shown in the drawings and described in detail herein, specific embodiments, with the understanding that the present disclosure is to be considered as an exemplification of the principles of the invention, and is not intended to limit the invention to the embodiment(s) illustrated.

The invention and accompanying drawings will now be discussed in reference to the numerals provided therein to enable one skilled in the art to practice the present invention. The drawings and descriptions are exemplary of various aspects of the invention and are not intended to narrow the scope of the appended claims. Unless specifically noted, it is intended that the words and phrases in the specification and the claims be given their plain, ordinary and accustomed meaning to those of ordinary skill in the applicable arts. It is noted that the inventors can be their own lexicographers. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventor's intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112(f) or pre-AIA 35 U.S.C. § 112˜6. Thus, the use of the words “function,” “means” or “step” in the Detailed Description of the Invention or claims is not intended to somehow indicate a desire to invoke the special provisions of 35 U.S.C. § 112(f) or pre-AIA 35 U.S.C. § 112˜6 to define the invention. To the contrary, if the provisions of 35 U.S.C. § 112(f) or pre-AIA 35 U.S.C. § 112˜6 are sought to be invoked to define the inventions, the claims will specifically and expressly state the exact phrases “means for” or “step for” and the specific function (e.g., “means for roasting”), without also reciting in such phrases any structure, material or act in support of the function. Thus, even when the claims recite a “means for . . . ” or “step for . . . ” if the claims also recite any structure, material or acts in support of that means or step, or that perform the recited function, then it is the clear intention of the inventor not to invoke the provisions of 35 U.S.C. § 112(f) or pre-AIA 35 U.S.C. § 112˜6. Moreover, even if the provisions of 35 U.S.C. § 112(f) or pre-AIA 35 U.S.C. § 112˜6 are invoked to define the claimed inventions, it is intended that the inventions not be limited only to the specific structure, material or acts that are described in the illustrated embodiments, but in addition, include any and all structures, materials or acts that perform the claimed function as described in alternative embodiments or forms of the invention, or that are well known present or later-developed, equivalent structures, material or acts for performing the claimed function.

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and apparatus are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, apparatus and technologies to which the disclosed inventions may be applied. Thus, the full scope of the inventions is not limited to the examples that are described below.

Various aspects of the present invention may be described in terms of functional block components and various processing steps. Such functional blocks may be realized by any number of hardware or software components configured to perform the specified functions and achieve the various results.

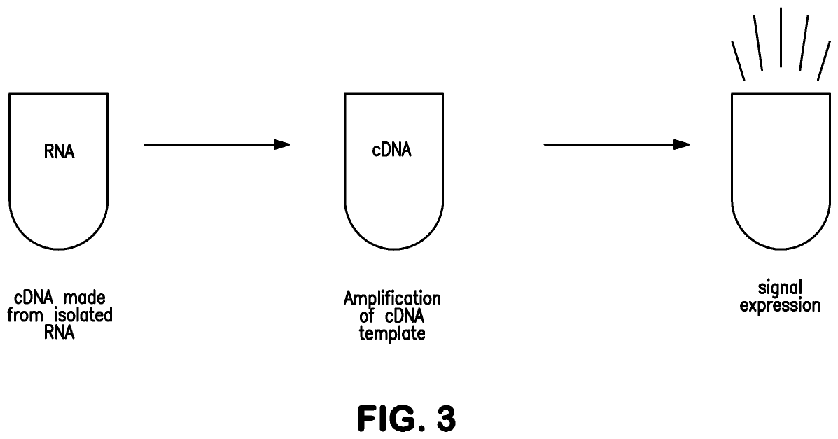

FIG. 3 illustrates schematically an exemplary laboratory procedure, specifically, the reverse transcription polymerase chain reaction (RT-qPCR), which has application in a wide variety of fields, for example, to determine whether a particular gene or gene sequence may be present in a tissue or fluid sample. This has particular application in determining whether, for example, a particular DNA, belonging to a virus or other pathogen, may be present in a blood sample taken from an individual who is suspected to have been exposed to a particular virus or other pathogen of interest.

It is to be understood that FIG. 3 is a schematic illustration of the steps in the method. However, in practical real-world examples, these method steps are typically, if not universally, being performed within the same test tube or other vessel, with the constituents or precursors thereof, all present simultaneously from the beginning of the procedure, notwithstanding the fact that in some such procedures it is already known that the early presence of certain of these constituents or precursors thereof may actually interfere with the complete and efficient performance of later steps within the procedure.

The RT-qPCR procedure begins with the acquisition of cDNA, from isolated RNA sample material (FIG. 3, left). The RNA is suspended within any suitable medium, pH buffered salt solution, together with appropriate reagents, including sequence specific oligonucleotide primers, reverse transcriptase enzyme and deoxynucleotides (dNTPs) Adenine, Cytosine, Guanine and Thymine triphosphates and heated to 42° C. and held at that temperature for 20 minutes. To inactivate the reverse transcriptase, the tube and fluid are then heated to 95° C. and held for 3 minutes. Both reverse transcription and PCR are performed in the same buffered salt solution with the only difference being the respective enzymes and initial product (RNA versus cDNA)

Amplification of the cDNA template (FIG. 3, center), and activation of the polymerase is then accomplished by heating to 95° C. for 3 minutes, followed by a cycle of 40 iterations of 95° C. for 3 seconds, followed by lowering to and holding at, 60° C. for 30 seconds.

Upon completion of the cycle, the presence or absence of the target RNA is revealed (FIG. 3, right) by fluorescence, which only occurs if the original target RNA is present.

In an optimized testing environment, the initial step in the left of FIG. 3 would be performed in a first test volume (first test tube). Then, once that step has been completed, the resultant material would be introduced into a separate new second test volume (second test tube), together with the reagents necessary to perform the second stage of the procedure. However, such division of steps and multiplication of test volumes inherently increases the number of units of equipment needed and necessarily increases the amount of time required to perform the tests.

As previously discussed in the Background section above, the reactions in the left and center portions of FIG. 3 require some of the same reactive material (nucleotide substrate). As such, there has been a trend toward employing the same common test volume (e.g., an undivided test tube volume), with all of the necessary reagents and/or test constituents or precursors thereof, present in the same tube at the start. Because of the competition between the two reactions for some of the same reagent materials, it is believed that there is a resultant loss of sensitivity of the overall procedure on the order of approximately thirty percent (30%).

To address this loss of sensitivity while seeking to maintain efficiencies of time and economy of material, the invention of the instant disclosure is directed to a multi-chambered test volume/container (e.g., test tube), that enables all of the necessary test constituents to be assembled together in a common tube, while preventing undesired interactions between same, until a specified predetermined stage in the procedure.

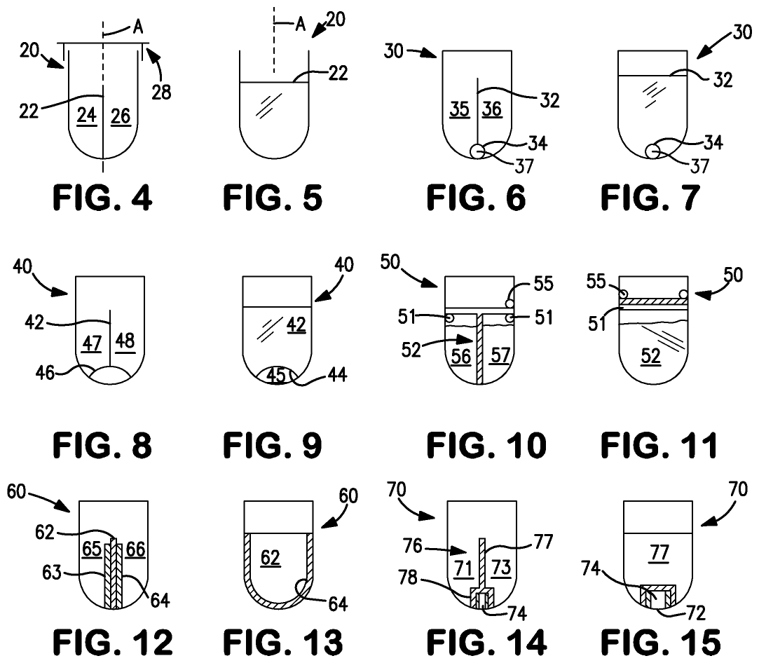

FIG. 4 is a schematic cross-sectional view of a test tube 20 according to an embodiment of the invention. FIG. 5 is a schematic cross-sectional view of the test tube 20 according to the embodiment of FIG. 4, rotated ninety degrees about a vertical axis from the view of FIG. 4. Test tube 20 may be fabricated from polypropylene or any other material suitable for medical or biological laboratory procedures. A septum 22 is positioned within tube 20 to divide an internal volume of tube 20 into two chambers 24, 26. An optically-clear top or cap 28 (which may be employed with all of the embodiments described herein) may be employed to both contain the contents during the test procedure, and to allow for visual observation and recording of potential fluorescence which may occur. Septum 22 may extend a complete height of the internal volume of tube 20, though it is contemplated that this is not essential for procedures in which the reactants are not volatile in any manner significant to the outcome of the test. In an embodiment of the invention, septum 22 is fabricated from paraffin wax, although one skilled in the art will recognize that other materials may be employed. Paraffin is advantageous because it has a melting point of approximately 95° C.

Thus, in a test procedure employing tube 20, the reactants necessary to perform the first stage of the procedure of FIG. 3 (left) may be placed in chamber 24, and the reactants for the second stage of the procedure of FIG. 3 (center), less the cDNA being created in the first stage, will be placed in chamber 26.

Septum 22, which may be held in place by a combination of friction and adhesion to inner surfaces of tube 20, will be begin to fail as the end of the first step in the procedure of FIG. 3, namely, at creation of the cDNA and as inactivation of reverse transcriptase, is being completed. Septum 22, after prolonged exposure to the high temperature, softens and melts, forming one or more holes or breaches, thus allowing chambers 24, 26 to come into liquid communication with one another, thus allowing the second step of the procedure (FIG. 3, center) to begin and be completed. It is recognized that while the test procedure is being performed, at one or more points during the procedure, the test tube may be placed on a shaker table, in a centrifuge, or other apparatus to facilitate the intermingling of the liquids in the respective chambers.

FIG. 6 is a schematic cross-sectional view of a test tube 30 according to an embodiment of the invention. FIG. 7 is a schematic cross-sectional view of test tube 30 according to the embodiment of FIG. 6, orthogonal to the view of FIG. 6. Tube 30 provided with a fixed septum 32, which may be fabricated from the same material as tube 30 or any other suitable material that will remain solid and imperforate throughout the temperature range of the procedure being performed. A window or aperture 34 is disposed in septum 32, for example, but not limited to, a position at the bottommost juncture of septum 32 and tube 30. Septum 32 divides an inner volume of tube 30 into chambers 35 and 36. Within window 34, a bead 37 of paraffin wax, or similar suitable material is disposed, acting as a plug to close off liquid communication between chambers 35 and 36. Similarly to the embodiment of FIGS. 3-4, during use, the appropriate reactants may be placed in respective chambers 35, 36, and as the first stage of the test progresses, bead 37 will melt, clearing window 34, and allowing the liquid materials within the two chambers to communicate and intermix.

FIG. 8 is a schematic cross-sectional view of a test tube 40 according to an embodiment of the invention. FIG. 9 is a schematic cross-sectional view of test tube 40 according to the embodiment of FIG. 8, orthogonal to the view of FIG. 8. Similar to the embodiment of FIGS. 6-7, tube 40 is provided with fixed septum 42, having an aperture, or in the illustrated embodiment, a recess 44, which creates a window 45 between a lower edge of septum 42 and a bottom inner surface of tube 40. Tube 40 and septum 42 may be fabricated from polypropylene or similar suitable material. A membrane 46 is positioned between the edge defining recess 44 and the bottom inner surface of tube 40. Membrane 46 is attached to tube 40, thus entrapping air or other suitable gas, between membrane 46 and tube 40. Membrane 46 is not, however, attached to, or fabricated from a material having proclivity to adhere to, septum 42. Thus, together, septum 42 and membrane 46 separate the internal volume of tube 40 into two chambers, 47 and 48. One skilled in the art will appreciate that by appropriate selection of the materials for membrane 46, its thickness, and the type and pressure of gas entrapped between membrane 46 and tube 40, that membrane 46 can be suitably configured to rupture upon exposure of tube 46 to a sustained predetermined temperature, such as at 95° C., in accordance with the procedure of FIG. 3. Upon rupture of membrane 46, such as at the end of the first stage of the procedure of FIG. 3, membrane 46, which preferably has a density and specific gravity greater than the liquid, will fall away from the lower edge of septum 42 defining recess 44, thus allowing chambers 47, 48 to be in liquid communication.

FIG. 10 is a schematic cross-sectional view of a test tube 50 according to an embodiment of the invention. FIG. 11 is a schematic cross-sectional view of test tube 50 according to the embodiment of FIG. 10, orthogonal to the view of FIG. 10. Tube 50 includes a removable septum 52. Tube 50 and septum 52 may be fabricated from polypropylene or any suitable material. Septum 52 includes a top 53 and a vertical portion 54. One or more expandable chambers 51 are positioned around the inner surface of tube 50, defining a position wherein septum 52 can be inserted, such that a bottom edge region of wall 54 defines a liquid-tight seal against the bottom inner surface of tube 50. Chamber(s) 51 is/are configured to expand when tube 50 is heated. A smaller, flexible retaining rim or bead 55 is disposed on an inner surface of tube 50, such that septum 52 divides an internal volume of tube 50 into chambers 56, 57. Top 53 will be provided with openings, or thin regions through which a syringe or needle may be inserted, on either side of vertical wall 54, to enable the introduction of the reagent materials In use, septum 52 is snapped into place, and the appropriate reagents will be placed in chambers 56, 57, and During the procedure, in accordance with the suitably selected material parameters, at a predetermined time during the procedure, such as at the end of the first stage of procedure FIG. 3, the gas will become sufficiently heated and expand, causing chamber(s) 51 to expand, and push top 53 past bead 55, raising a lowermost edge of vertical portion 54 away from the bottom of tube 50, causing chambers 56, 57 to come into liquid communication.

FIG. 12 is a schematic cross-sectional view of a test tube 60 according to an embodiment of the invention. FIG. 13 is a schematic cross-sectional view of the test tube according to the embodiment of FIG. 12, orthogonal to the view of FIG. 12. In this embodiment, tube 60 is provided with beads 63, 64, configured to frictionally receive between them septum 62, to divide the internal volume of tube 60 into chambers 65, 66. Tube 60 and beads 63, 64 may be fabricated from polypropylene, or other similar suitable material. Septum 62 is preferably fabricated from a material having a lower coefficient of thermal expansion than that of tube 60 and/or beads 63, 64, such that, upon continued exposure to a temperature at or above a predetermined temperature, as described above with respect to foregoing embodiments, tube 60, and beads 63, 64, will expand, thus releasing grip on septum 62. Preferably, septum 62 will have a density or specific gravity that is less than that of the liquid in chambers 65, 66, so that septum 62 will be prompted to be floated upwardly from the bottom of tube 60, thus allowing chambers 65, 66 to communicate.

FIG. 14 is a schematic cross-sectional view of a test tube 70 according to an embodiment of the invention. FIG. 15 is a schematic cross-sectional view of test tube 70 according to the embodiment of FIG. 14, orthogonal to the view of FIG. 14. Tube 70, which may be fabricated from polypropylene or any suitable material, has a hollow projection 72 with an upwardly-facing opening (relative to the illustration of FIGS. 14, 15), defining a volume 74. Septum 76 has a wall 77 and a fitting 78 having a shape that mates with an outer surface of projection 72, with a weak force-fit engagement. Septum 76, fitting 78 in particular, may be fabricated from a suitable material, having a greater coefficient of thermal expansion than the material from which tube 70 and/or projection 72 are fabricated. Also, septum 76 may be fabricated from a material having a lower density/specific gravity than the liquids which will be used in tube 70. In use, septum 76 is inserted into tube 70, such that projection 72 and fitting 78 engage with a weak frictional fit, and the appropriate reactants will be inserted into chambers 71, 73. The level of friction, and the volume enclosed by projection 72 and fitting 78 will be selected such that the pressure caused by the expanding gas will, at an appropriate time during the procedure, cause septum 76 to be popped off of projection 72, in a manner similar to that described with respect to the foregoing embodiments.

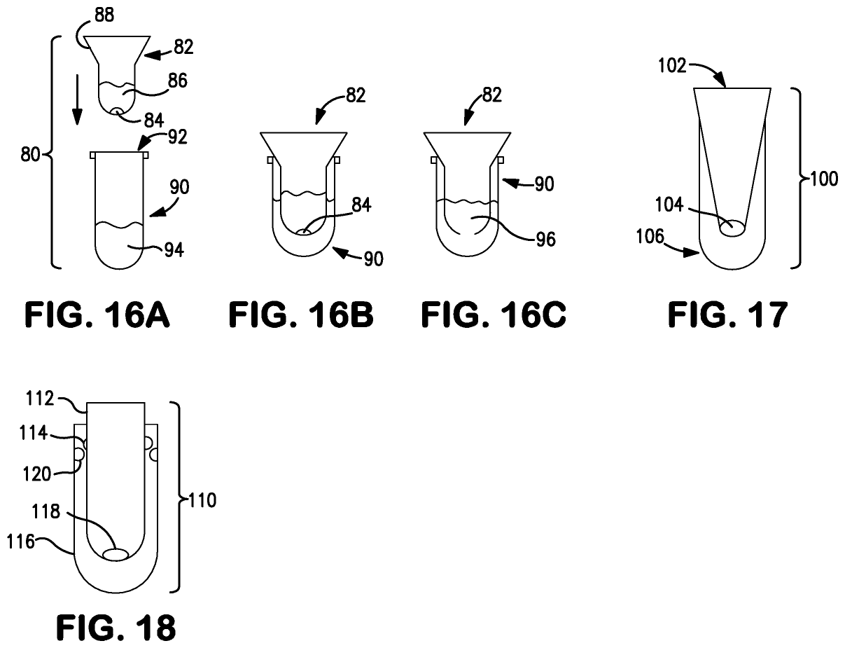

FIGS. 16A-16C, 17-18 illustrate variations on a further embodiment of the invention. In the embodiment of FIGS. 16A-16C, 17-18, the multi-chamber test tube comprises two distinct but cooperative components, namely: 1) an upper, or inner, chamber, wherein the breachable septum is incorporated; and 2) a lower, or outer chamber.

FIGS. 16A-16C illustrate a first variation of this embodiment. Multi-chamber test tube 80 comprises an upper/inner chamber 82, having a aperture 96 disposed in a bottom region thereof, filled with a bead 84 of wax or similar material, such as described hereinabove with respect to previous embodiments. Upper/inner chamber 82 further includes a flare or step 88, the purpose of which is described hereinafter. Suitable reagent material 86 is contained in chamber 82.

Test Tube 80 further includes lower/outer chamber 90, having an upper edge or rim 92, and which receives suitable reagent 94. The relative dimensions of chambers 82 and 90, and the cooperation of flare or step 88 with edge or rim 92, allow for the insertion of chamber 82 into chamber 90 (FIG. 16B), but leaving sufficient clearance between the bottoms of chambers 82 and 90, so that the reagent 94 in chamber 90 is not pushed out.

Once the chambers have been coupled, and the appropriate test materials have been placed in the respective chambers, as described hereinabove with respect to the prior embodiments, during the test procedure, heat will be applied, and the septum, defined by aperture 96 and bead 84, will be breached via the softening of bead 84 in response to the heat. Thus, reagent 84 will tend to flow at least partially out of chamber 82 (as indicated by the arrows in FIG. 16C), and reagents 86 and 94 will be combined, to permit the test to go forward.

FIG. 17 illustrates another variation of this embodiment, wherein multi-chamber test tube 100 comprises a substantially conical upper/inner chamber having a septum 104 (formed by a complementary aperture and wax bead, not separately numbered), which is insertingly received by a generally cylindrical lower/inner tube 106. In this variation, as with the variation illustrated in FIGS. 16A-16C, the respective chambers are dimensioned so that the bottom of chamber 102 is suspended above the bottom of chamber 106 a sufficient distance to allow for insertion without spillage of the reagent (not shown) contained therein. Operation of this variation is substantially the same as previously described.

FIG. 18 illustrates a further variation of this embodiment, wherein multi-chamber test tube 110 comprises upper/inner tube 112, having an intermittent or entirely circumferential rim or ridge 114, and septum 118, again, formed by a complementary aperture and wax bead (not separately numbered), which is insertingly received by a generally cylindrical lower/inner tube 116 having a radially inwardly projecting intermittent or entirely circumferential rim or ridge 120. In this variation, as with the variation illustrated in FIGS. 16A-16C, the respective chambers are dimensioned so that the bottom of chamber 112 is suspended above the bottom of chamber 116 a sufficient distance to allow for insertion without spillage of the reagent (not shown) contained therein. Operation of this variation is substantially the same as previously described.

A further variation of FIG. 18 is envisioned wherein one or the other of chambers 112, 118 is provided with a pair of vertically-spaced rims or ridges, fabricated of a resilient material, and configured to receive the single rim or ridge of the other therebetween, so that the two chambers may be “snapped” together to prevent undesired separation thereof.

For each of embodiments of FIGS. 16A-16C, 17-18, suitably configured caps may be provided to prevent loss of fluid, before, during or after any test is conducted. In addition, the materials from which the tube components of FIGS. 16A-16C, 17-18 may be fabricated from polypropylene, or any other suitable material, as previously described hereinabove.

Other mechanisms for achieving dislodgement of a septum separating two chambers are also contemplated. For example, in an embodiment similar to that of FIGS. 12-13, the tube, at the appropriate time during the procedure, may be subjected to vibration, such as ultrasonic vibration, to cause the separation of a frictionally-held septum from the interior wall of a test tube. Alternatively, a shaker table or similar device might be used to provide microvibration of the tube.

In another alternative embodiment, a test tube may be provided with a septum having a printed circuit board thereon, operating an electromagnetically operated microgate, to enable communication between chambers separated by the septum.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

In embodiments of this invention, the test tubes as described above may be configured as individual standalone tubes. Alternatively, they may be configured as multiples of tubes, arranged in arrays, possibly with lines of weakness arranged therebetween, wherein rows or blocks of such tubes may be broken off for use, in quantities as needed.

In the embodiments described herein, the test tubes have a single walled septum, dividing the test tube into two separate, fluidically-isolated chambers. In alternative embodiments, tubes may be provided having multi-wall or multi-component septa, such that the test tube may be divided into 3 or more chambers, with the mechanisms for breaching a wall between any two chambers configured to fail or otherwise permit communication as the same or at different times or under different conditions. For example, if different walls of a plurality of septa are configured to fail at different (e.g., rising) temperatures, then a plurality of walls may be configured to fail sequentially, as necessary or desired for a particular application.

While the term “test tube” is used herein to describe the several embodiments of the invention, it is to be understood that the principles of the present invention may be applied to a wide variety of laboratory type containers having a range of shapes and configurations. Accordingly, the term “test tube” is to be construed in the broadest possible context as simply referring to a container for use in a laboratory or other setting for assaying, sampling or other testing procedures.

Although the invention has been described with reference to the above examples, it will be understood that many modifications and variations are contemplated within the true spirit and scope of the embodiments of the invention as disclosed herein. Many modifications and other embodiments of the invention set forth herein will come to mind to one skilled in the art to which the invention pertains having the benefit of the teachings presented in the foregoing descriptions and the associated drawings. Therefore, it is to be understood that the invention shall not be limited to the specific embodiments disclosed and that modifications and other embodiments are intended and contemplated to be included within the scope of the appended claims. Although specific terms are employed herein, they are used in a generic and descriptive sense only and not for purposes of limitation.

What is claimed is:

1. A multi-chambered test container, comprising:
an outer shell, defining an inner volume;
a septum, disposable within the shell, having at least one wall defining at least two chambers within the inner volume;
a mechanism cooperatively engaged with at least one of the outer shell and the septum, which causes a change in relationship between the at least two chambers, such that in a first configuration, the at least two chambers are not in liquid communication to one another, and in a second configuration, the at least two chambers are in liquid communication with one another.

2. The multi-chambered test container according to claim 1, wherein the mechanism comprises the septum being fabricated from a material that will at least partially fail when the test container is exposed to at least one of a predetermined temperature and a predetermined pressure.

3. The multi-chambered test container according to claim 1, wherein the mechanism comprises the septum being moved from a first physical orientation relative to the outer shell, wherein the at least two chambers are not in liquid communication, to a second orientation, wherein the at least two chambers are in liquid communication, the movement of the septum occurring when the test container has been exposed to at least one of a predetermined temperature and a predetermined pressure.

4. The multi-chambered test container according to claim 1, wherein the mechanism comprises a plug, disposed in or adjacent to at least one of the septum and an inner wall of the outer shell, wherein the plug is fabricated from a material that will at least partially fail when the test container is exposed to at least one of a predetermined temperature and a predetermined pressure.

5. The multi-chambered test container according to claim 3, wherein the mechanism further comprises thermal expansion causing separation of an inner surface of the outer shell and the septum.

6. The multi-chambered test container according to claim 1, wherein the mechanism comprises a frangible membrane disposed between a lower edge of the septum and a bottom inner surface of the shell.

7. The multi-chambered test container according to claim 3, wherein the mechanism comprises an expandable chamber which expands upon application of heat to a predetermined temperature and exerts pressure on the septum to dislodge the septum at least partially away from an inner surface of the outer shell.

8. The multi-chambered test container according to claim 3, wherein the mechanism comprises exposing the test container to vibration to dislodge the septum at least partially away from an inner surface of the outer shell.

9. The multi-chambered test container according to claim 3, wherein the mechanism comprises the septum being fabricated from a material having a lower coefficient of thermal expansion than the material of the outer shell, such that upon exposure to heat above a predetermined temperature, the septum will become separated from the outer shell.

10. The multi-chambered test container according to claim 3, wherein the mechanism comprises a pocket, defined between mating portions of the septum and the inner surface of the outer shell, such that upon exposure to heat above a predetermined temperature, gas entrapped within the pocket expands and forces separation of the septum from the outer shell.

11. The multi-chambered test container according to claim 1, further comprising a removable optically-clear cap.

12. A method of performing a test, comprising:
providing a multi-chambered test container, comprising the steps of:
providing an outer shell, defining an inner volume;
providing a septum, disposable within the shell, having at least one wall defining at least two chambers within the inner volume;
providing a mechanism cooperatively engaged with at least one of the outer shell and the septum, which causes a change in relationship between the at least two chambers, such that in a first configuration, the at least two chambers are not in liquid communication to one another, and in a second configuration, the at least two chambers are in liquid communication with one another;
the method further comprising the steps of:
placing at least one first reactant within a first of the defined at least two chambers;
placing at least one second reactant with a second of the defined at least two chambers;
disposing the septum within the shell;
initiating a test procedure using the multi-chambered test container; and
actuating the mechanism.

13. The multi-chambered test container according to claim 1, wherein the septum comprises an inner shell, defining an inner shell inner volume, the inner shell being insertingly receivable within at least a portion of the outer shell; and the mechanism is cooperatively engaged with the inner shell.

14. The multi-chambered test container according to claim 14, wherein the mechanism comprises:
an aperture disposed in a generally-bottom region of the inner shell; and
a plug, disposed in or adjacent to the aperture, wherein the plug is fabricated from a material that will at least partially fail when the test container is exposed to at least one of a predetermined pressure and a predetermined temperature.

15. The multi-chambered test container according to claim 13, further comprising a mechanism for preventing the inner shell from bottoming out in the outer shell.

16. The multi-chambered test container according to claim 13, further comprising a mechanism for preventing undesired separation of the inner and outer shells, once the inner shell has been inserted into the outer shell.

17. The method according to claim 12, wherein the mechanism comprises the septum being fabricated from a material that will at least partially fail when the test container is exposed to at least one of a predetermined temperature and a predetermined pressure.

18. The method according to claim 12, wherein the mechanism comprises the septum being moved from a first physical orientation relative to the outer shell, wherein the at least two chambers are not in liquid communication, to a second orientation, wherein the at least two chambers are in liquid communication, the movement of the septum occurring when the test container has been exposed to at least one of a predetermined temperature and a predetermined pressure.

19. The method according to claim 12, wherein the mechanism comprises a plug, disposed in or adjacent to at least one of the septum and an inner wall of the outer shell, wherein the plug is fabricated from a material that will at least partially fail when the test container is exposed to at least one of a predetermined temperature and a predetermined pressure.

20. The method according to claim 18, wherein the mechanism further comprises thermal expansion causing separation of an inner surface of the outer shell and the septum.
